(19) United States
(12) Patent Application Publication
(10) Pub. No.: US 2003/0181473 A1
(43) Pub. Date: Sep. 25, 2003

(54) Farnesyl protein transferase inhibitor combinations with taxane compounds

(76) Inventors: Peter Albert Palmer, Keerbergen (BE); Ivan David Horak, Yardley, PA (US)

Correspondence Address: Philip S Johnson, Johnson & Johnson, One Johnson & Johnson Plaza, New Brunswick, NJ, 08933-7003, US

(21) Appl. No.: 10/220,396

(22) PCT Filed: Feb. 26, 2001
(86) PCT No.: PCT/EP01/02170

(30) Foreign Application Priority Data
Feb. 29, 2000 (EP) 00200689.8

(51) Int. Cl. A61K 31/517; A61K 31/4745; A61K 31/4709; A61K 31/337

(52) U.S. Cl. 514/266.31; 514/312; 514/291; 514/292; 514/314; 514/449

(57) ABSTRACT

The present invention is concerned with combinations of a farnesyl transferase inhibitor and a taxane compound for inhibiting the growth of tumor cells and useful in the treatment of cancer.

The present invention is concerned with combinations of a farnesyl transferase inhibitor and a taxane compound for inhibiting the growth of tumor cells, useful in the treatment of cancer.

Oncogenes frequently encode protein components of signal transduction pathways which lead to stimulation of cell growth and mitogenesis. Oncogene expression in cultured cells leads to cellular transformation, characterized by the ability of cells to grow in soft agar and the growth of cells as dense foci lacking the contact inhibition exhibited by non-transformed cells. Mutation and/or overexpression of certain oncogenes is frequently associated with human cancer. A particular group of oncogenes is known as ras which have been identified in mammals, birds, insects, mollusks, plants, fungi and yeasts. The family of mammalian ras oncogenes consists of three major members (“isoforms”): H-ras, K-ras and N-ras oncogenes. These ras oncogenes code for highly related proteins generically known as p21^{ras}. Once attached to plasma membranes, the mutant or oncogenic forms of p21^{ras} will provide a signal for the transformation and uncontrolled growth of malignant tumor cells. To acquire this transforming potential, the precursor of the p21^{ras} oncoprotein must undergo an enzymatically catalyzed farnesylation of the cysteine residue located in a carboxyl-terminal tetrapeptide. Therefore, inhibitors of the enzyme that catalyzes this modification, farnesyl protein transferase, will prevent the membrane attachment of p21^{ras} and block the aberrant growth of ras-transformed tumors. Hence, it is generally accepted in the art that farnesyl transferase inhibitors can be very useful as anticancer agents for tumors in which ras contributes to transformation.

Since mutated, oncogenic forms of ras are frequently found in many human cancers, most notably in more than 50% of colon and pancreatic carcinomas (Kohl et al., Science, vol 260, 1834-1837, 1993), it has been suggested that farnesyl tranferase inhibitors can be very useful against these types of cancer. Following further investigations, it has been found that a farnesyl transferase inhibitor is capable of demonstrating antiproliferative effects in vitro and antitumor effects in vivo in a variety of human tumor cell lines with and without ras gene mutations.

WO-97/21701 describes the preparation, formulation and pharmaceutical properties of farnesyl protein transferase inhibiting (imidazoly-5-yl)methyl-2-quinolinone derivatives of formulas (I), (II) and (M), as well as intermediates of formula (II) and (TR) that are metabolized in vivo to the compounds of formula (I). The compounds of formulas (I), (II) and (III) are represented by

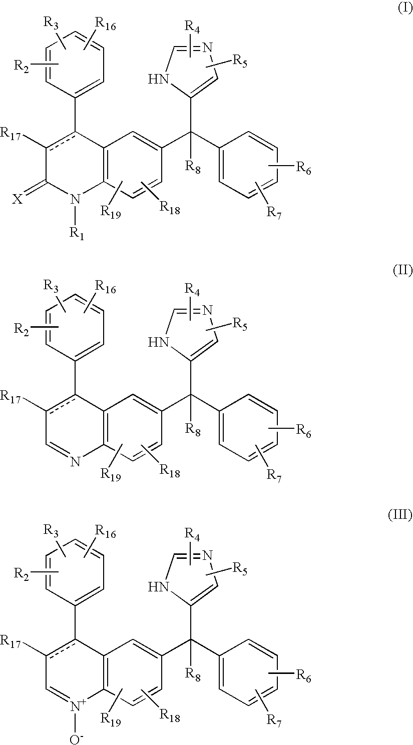

the pharmaceutically acceptable acid or base addition salts and the stereochemically isomeric forms thereof, wherein

the dotted line represents an optional bond;

X is oxygen or sulfur;

R¹ is hydrogen, C₁₋₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,

or a radical of formula -Alk¹-C(═O)—R⁹, -Alk¹-S(O)—R⁹ or -Alk¹-S(O)₂—R⁹,

wherein Alk¹ is C₁₋₆alkanediyl,

R⁹ is hydroxy, C₁₋₆alkyl, C₁₋₆alkyloxy, amino, C₁₋₈alkylamino or C₁₋₈alkylamino substituted with C₁₋₆alkyloxycarbonyl;

R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyl-oxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or

when on adjacent positions R² and R³ taken together may form a bivalent radical of formula

| | |
|---|---|
| —O—CH₂—O— | (a-1), |
| —O—CH₂—CH₂—O— | (a-2), |
| —O—CH═CH— | (a-3), |
| —O—CH₂—CH₂— | (a-4), |
| —O—CH₂—CH₂—CH₂— | (a-5), or |
| —CH═CH—CH═CH— | (a-6); |

R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;

R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or

when on adjacent positions R⁶ and R⁷ taken together may form a bivalent radical of formula

| | |
|---|---|
| —O—CH₂—O— | (c-1), or |
| —CH═CH—CH═CH— | (c-2); |

R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)-aminoC₁₋₆alkyl, imidazolyl, haloC₁₋₆alkyl, C₁₋₆alkyloxyC₁₋₆alkyl, aminocarbonylC₁₋₆alkyl, or a radical of formula

| | |
|---|---|
| —O—R¹⁰ | (b-1), |
| —S—R¹⁰ | (b-2), |
| —N—R¹¹R¹² | (b-3), |

wherein

R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;

R¹¹ is hydrogen, C₁₋₁₂alkyl, Ar¹ or Ar²C₁₋₆alkyl;

R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonyl-C₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk²-OR¹³ or -Alk²-NR¹⁴R¹⁵;

wherein Alk² is C₁₋₆alkanediyl;

R¹³ is hydrogen, C₁₋₆alkyl, C₁₋₆alkylcarbonyl, hydroxy-C₁₋₆alkyl, Ar¹ or Ar²C₁₋₆alkyl;

R¹⁴ is hydrogen, C₁₋₆alkyl, Ar¹ or Ar²C₁₋₆alkyl;

R¹⁵ is hydrogen, C₁₋₆alkyl, C₁₋₆alkylcarbonyl, Ar¹ or Ar²C₁₋₆alkyl;

R¹⁷ is hydrogen, halo, cyano, C₁₋₆alkyl, C₁₋₆alkyloxycarbonyl, Ar¹;

R¹⁸ is hydrogen, C₁₋₆alkyl, C₁₋₆alkyloxy or halo;

R¹⁹ is hydrogen or C₁₋₆alkyl;

Ar¹ is phenyl or phenyl substituted with C₁₋₆alkyl, hydroxy, amino, C₁₋₆alkyloxy or halo; and

Ar² is phenyl or phenyl substituted with C₁₋₆alkyl, hydroxy, amino, C₁₋₆alkyloxy or halo.

WO-97/16443 concerns the preparation, formulation and pharmaceutical properties of farnesyl protein transferase inhibiting compounds of formula (IV), as well as intermediates of formula (V) and (VI) that are metabolized in vivo to the compounds of formula (IV). The compounds of formulas (IV), (V) and (VI) are represented by

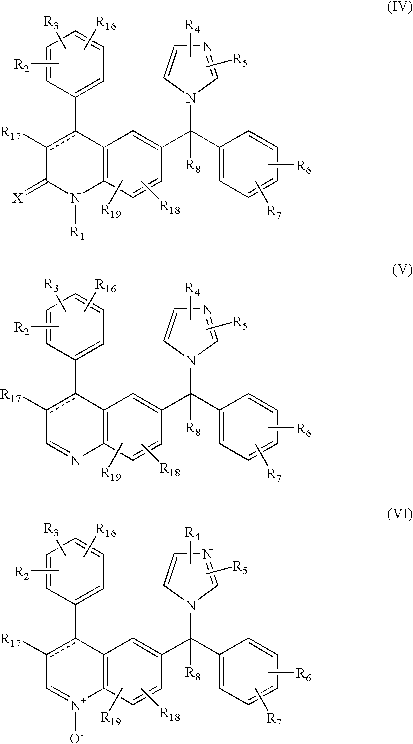

the pharmaceutically acceptable acid or base addition salts and the stereochemically isomeric forms thereof, wherein

the dotted line represents an optional bond;

X is oxygen or sulfur;

R¹ is hydrogen, C₁₋₁₂alkyl, Ar¹, Ar²C₁₋₆alkyl, quinolinylC₁₋₆alkyl pyridyl-C₁₋₆alkyl, hydroxyC₁₋₆alkyl, C₁₋₆alkyloxyC₁₋₆alkyl, mono- or di(C₁₋₆alkyl)-aminoC₁₋₆alkyl, aminoC₁₋₆alkyl,

or a radical of formula -Alk¹-C(═O)—R⁹, -Alk¹-S(O)—R⁹ or -Alk¹-S(O)₂—R⁹,

wherein Alk¹ is C₁₋₆alkanediyl,

R⁹ is hydroxy, C₁₋₆alkyl, C₁₋₆alkyloxy, amino, C₁₋₈alkylamino or C₁₋₈alkylamino substituted with C₁₋₆alkyloxycarbonyl;

R² and R³ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, amino-C₁₋₆alkyloxy, mono- or di (C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl; or

when on adjacent positions R² and R³ taken together may form a bivalent radical of formula

| | |
|---|---|
| —O—CH₂—O— | (a-1), |
| —O—CH₂—CH₂—O— | (a-2), |
| —O—CH═CH— | (a-3), |
| —O—CH₂—CH₂— | (a-4), |
| —O—CH₂—CH₂—CH₂— | (a-5), or |
| —CH═CH—CH═CH— | (a-6); |

R⁴ and R⁵ each independently are hydrogen, Ar¹, C₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₆alkyl;

R⁶ and R⁷ each independently are hydrogen, halo, cyano, C₁₋₆alkyl, C₁₋₆alkyloxy or Ar²oxy;

R⁸ is hydrogen, C₁₋₆alkyl, cyano, hydroxycarbonyl, C₁₋₆alkyloxycarbonyl, C₁₋₆alkyl-carbonylC₁₋₆alkyl, cyanoC₁₋₆alkyl, C₁₋₆alkyloxycarbonylC₁₋₆alkyl, hydroxy-carbonylC₁₋₆alkyl, hydroxyC₁₋₆alkyl, aminoC₁₋₆alkyl, mono- or di(C₁₋₆alkyl)-aminoC₁₋₆alkyl, haloC₁₋₆alkyl, C₁₋₆alkyloxyC₁₋₆alkyl, aminocarbonylC₁₋₆alkyl, Ar¹, Ar²C₁₋₆alkyioxyC₁₋₆alkyl, C₁₋₆alkylthioC₁₋₆alkyl;

R¹⁰ is hydrogen, C₁₋₆alkyl, C₁₋₆alkyloxy or halo;

R¹¹ is hydrogen or C₁₋₆alkyl;

Ar¹ is phenyl or phenyl substituted with C₁₋₆alkyl, hydroxy, amino, C₁₋₆akyloxy or halo;

Ar² is phenyl or phenyl substituted with C₁₋₆alkyl, hydroxy, amino, C₁₋₆alkyloxy or halo.

WO-98/40383 concerns the preparation, formulation and pharmaceutical properties of farnesyl protein transferase inhibiting compounds of formula (VII)

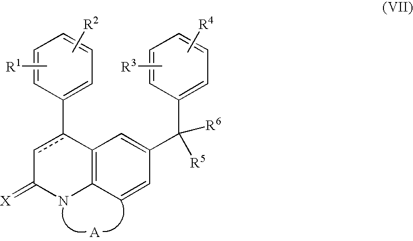

the pharmaceutically acceptable acid addition salts and the stereochemically isomeric forms thereof, wherein

the dotted line represents an optional bond;

X is oxygen or sulfur;

-A- is a bivalent radical of formula

| | | | |
|---|---|---|---|
| —CH═CH— | (a-1), | —CH₂—S— | (a-6), |
| —CH₂—CH₂— | (a-2), | —CH₂—CH₂—S— | (a-7), |
| —CH₂—CH₂—CH₂— | (a-3), | —CH═N— | (a-8), |
| —CH₂—O— | (a-4), | —N═N— | (a-9), or |
| —CH₂—CH₂—O— | (a-5), | —CO—NH— | (a-10); |

wherein optionally one hydrogen atom may be replaced by C₁₋₄alkyl or Ar¹;

R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar², Ar²-C₁₋₆alkyl, Ar²-oxy, Ar²-C₁₋₆alkyloxy; or when on adjacent positions R¹ and R² taken together may form a bivalent radical of formula

| | |
|---|---|
| —O—CH₂—O— | (b-1), |
| —O—CH₂—CH₂—O— | (b-2), |
| —O—CH═CH— | (b-3), |
| —O—CH₂—CH₂— | (b-4), |
| —O—CH₂—CH₂—CH₂— | (b-5), or |
| —CH═CH—CH═CH— | (b-6); |

R³ and R⁴ each independently are hydrogen, halo, cyano, C₁₋₆alkyl, C₁₋₆alkyloxy, Ar³-oxy, C₁₋₆alkylthio, di(C₁₋₆alkyl)amino, trihalomethyl, trihalomethoxy, or when on adjacent positions R³ and R⁴ taken together may form a bivalent radical of formula

| | |
|---|---|
| —O—CH₂—O— | (c-1), |
| —O—CH₂—CH₂—O— | (c-2), or |
| —CH═CH—CH═CH— | (c-3); |

R⁵ is a radical of formula

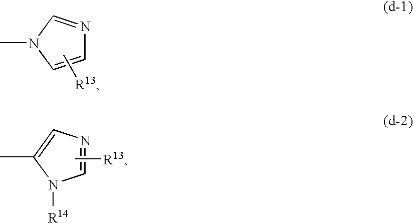

wherein

R¹³ is hydrogen, halo, Ar⁴, C₁₋₆alkyl, hydroxyC₁₋₆alkyl. C₁₋₆alkyloxy-C₁₋₆alkyl, C₁₋₆alkyloxy, C₁₋₆alkylthio, amino, C₁₋₆alkyloxy-carbonyl, C₁₋₆alkyS(O)C₁₋₆alkyl or C₁₋₆alkylS(O)2C₁₋₆alkyl;

R¹⁴ is hydrogen, C₁₋₆alkyl or di(C₁₋₄alkyl)aminosulfonyl;

R⁶ is hydrogen, hydroxy, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar⁵, Ar⁵-C₁₋₆alkyloxyC₁₋₆alkyl; or a radical of formula

—O—R⁷ (e-1),

—S—R⁷ (e-2),

—N—R⁸R⁹ (e-3),

wherein

R⁷ is hydrogen, C₁₋₆alkyl, C₁₋₆alkylcarbonyl, Ar⁶, Ar⁶-C₁₋₆alkyl, C₁₋₆alkyloxycarbonylC₁₋₆alkyl, or a radical of formula -Alk-OR¹⁰ or -Alk-NR¹¹R¹²;

R⁸ is hydrogen, C₁₋₆alkyl, Ar⁷ or Ar⁷-C₁₋₆alkyl;

R⁹ is hydrogen, C₁₋₆alkyl, C₁₋₆alkylcarbonyl, C₁₋₆alkyloxycarbonyl, C₁₋₆alkylaminocarbonyl, Ar⁸, Ar⁸-C₁₋₆alkyl, C₁₋₆alkylcarbonyl-C₁₋₆alkyl, Ar⁸-carbonyl, Ar⁸-C₁₋₆alkylcarbonyl, aminocarbonyl-carbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino,

or a radical or formula -Alk-OR¹⁰ or -Alk-NR¹¹R¹²;

wherein Alk is C₁₋₆alkanediyl;

R¹⁰ is hydrogen, C₁₋₆alkyl, C₁₋₆alkylcarbonyl, hydroxyC₁₋₆alkyl, Ar⁹ or Ar⁹-C₁₋₆alkyl;

R¹¹ is hydrogen, C₁₋₆alkyl, C₁₋₆alkylcarbonyl, Ar¹⁰ or Ar¹⁰-C₁₋₆alkyl;

R¹² is hydrogen, C₁₋₆alkyl, Ar¹¹ or Ar¹¹-C₁₋₆alkyl; and

Ar¹ to Ar¹¹ are each independently selected from phenyl; or phenyl substituted with halo, C₁₋₆alkyl, C₁₋₆alkyloxy or trifluoromethyl.

WO-98/49157 concerns the preparation, formulation and pharmaceutical properties of farnesyl protein transferase inhibiting compounds of formula (VIII)

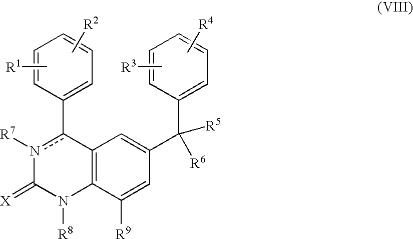

the pharmaceutically acceptable acid addition salts and the stereochemically isomeric forms thereof, wherein

the dotted line represents an optional bond;

X is oxygen or sulfur;

R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar¹, Ar¹C₁₋₆alkyl, Ar¹oxy or Ar¹C₁₋₆alkyloxy;

R³ and R⁴ each independently are hydrogen, halo, cyano, C₁₋₆alkyl, C₁₋₁₆alkyloxy, Ar¹oxy, C₁₋₆alkylthio, di(C₁₋₆alkyl)amino, trihalomethyl or trihalomethoxy;

R⁵ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar¹, Ar¹C₁₋₆alkyloxyC₁₋₆alkyl; or a radical of formula

| | |
|---|---|
| —O—R¹⁰ | (a-1), |
| —S—R¹⁰ | (a-2), |
| —N—R¹¹R¹² | (a-3), |

wherein

R¹⁰ is hydrogen, C₁₋₆alkyl, C₁₋₆alkylcarbonyl, Ar¹, Ar¹C₁₋₆alkyl, C₁₋₆alkyloxycarbonylC₁₋₆alkyl, or a radical of formula -Alk-OR¹³ or -Alk-NR¹⁴R¹⁵;

R¹¹ is hydrogen, C₁₋₆alkyl, Ar¹ or Ar¹C₁₋₆alkyl;

R¹² is hydrogen, C₁₋₆alkyl, C₁₋₆alkylcarbonyl, C₁₋₆alkyloxycarbonyl, C₁₋₆alkylaminocarbonyl, Ar¹, Ar¹C₁₋₆alkyl, C₁₋₆alkylcarbonyl-C₁₋₆alkyl, Ar¹carbonyl, Ar¹C₁₋₆alkylcarbonyl, aminocarbonyl-carbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonyl, amino,

or a radical or formula -Alk-OR¹³ or -Alk-NR¹⁴R¹⁵;

wherein Alk is C₁₋₆alkanediyl;

R¹³ is hydrogen, C₁₋₆alkyl, C₁₋₆alkylcarbonyl, hydroxy-C₁₋₆alkyl, Ar¹ or Ar¹C₁₋₆alkyl;

R¹⁴ is hydrogen, C₁₋₆alkyl, Ar¹ or Ar¹C₁₋₆alkyl;

R¹⁵ is hydrogen, C₁₋₆alkyl, C₁₋₆alkylcarbonyl, Ar¹ or Ar¹C₁₋₆alkyl;

R⁶ is a radical of formula

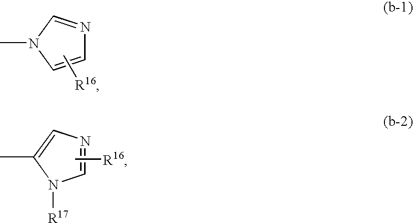

wherein

R¹⁶ is hydrogen, halo, Ar¹, C₁₋₆alkyl, hydroxyC₁₋₆alkyl, C₁₋₆alkyloxy-C₁₋₆alkyl, C₁₋₆alkyloxy, C₁₋₆alkylthio, amino, C₁₋₆alkyloxycarbonyl, C₁₋₆alkylthioC₁₋₆alkyl, C₁₋₆alkylS(O)C₁₋₆alkyl or C₁₋₆alkylS(O)2C₁₋₆alkyl;

R¹⁷ is hydrogen, C₁₋₆alkyl or di(C₁₋₄alkyl)aminosulfonyl;

R⁷ is hydrogen or C₁₋₆alkyl provided that the dotted line does not represent a bond;

R⁸ is hydrogen, C₁₋₆alkyl or Ar²CH₂ or Het¹CH₂;

R⁹ is hydrogen, C₁₋₆alkyl, C₁₋₆alkyloxy or halo; or

R⁸ and R⁹ taken together to form a bivalent radical of formula

| | |
|---|---|
| —CH═CH— | (c-1), |
| —CH₂—CH₂— | (c-2), |
| —CH₂—CH₂—CH₂— | (c-3), |
| —CH₂—O— | (c-4), or |
| —CH₂—CH₂—O— | (c-5); |

Ar¹ is phenyl; or phenyl substituted with 1 or 2 substituents each independently selected from halo, C₁₋₆alkyl, C₁₋₆alkyloxy or trifluoromethyl;

Ar² is phenyl; or phenyl substituted with 1 or 2 substituents each independently selected from halo, C₁₋₆alkyl, C₁₋₆alkyloxy or trifluoromethyl; and

Het¹ is pyridinyl; pyridinyl substituted with 1 or 2 substituents each independently selected from halo, C₁₋₆alkyl, C₁₋₆alkyloxy or trifluoromethyl.

WO-00/39082 concerns the preparation, formulation and pharmaceutical properties of farnesyl protein transferase inhibiting compounds of formula (IX)

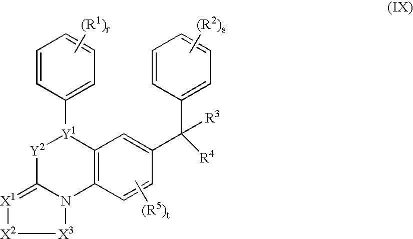

or the pharmaceutically acceptable acid addition salts and the stereochemically isomeric forms thereof, wherein

═X¹—X²—X³— is a trivalent radical of formula

| | | | |
|---|---|---|---|
| ═N—CR⁶═CR⁷— | (x-1), | ═CR⁶—CR⁷═CR⁸— | (x-6), |
| ═N—N═CR⁶— | (x-2), | ═CR⁶—N═CR⁷— | (x-7), |
| ═N—NH—C(═O)— | (x-3), | ═CR⁶—NH—C(═O)— | (x-8), or |
| ═N—N═N— | (x-4), | ═CR⁶—N═N— | (x-9); |
| ═N—CR⁶═N— | (x-5), | | |

wherein each R⁶, R⁷ and R⁸ are independently hydrogen, C₁₋₄alkyl, hydroxy, C₁₋₄alkyloxy, aryloxy, C₁₋₄alkyloxycarbonyl, hydroxyC₁₋₄alkyl, C₁₋₄alkyloxyC₁₋₄alkyl, mono- or di(C₁₋₄alkyl)aminoC₁₋₄alkyl, cyano, amino, thio, C₁₋₄alkylthio, arylthio or aryl;

>Y¹—Y²— is a trivalent radical of formula

| | |
|---|---|
| >CH—CHR⁹— | (y-1), |
| >C═N— | (y-2), |
| >CH—NR⁹— | (y-3),or |
| >C═CR⁹— | (y-4); |

wherein each R⁹independently is hydrogen, halo, halocarbonyl, aminocarbonyl, hydroxyC₁₋₄alkyl, cyano, carboxyl, C₁₋₄alkyl, C₁₋₄alkyloxy, C₁₋₄alkyloxyC₁₋₄alkyl, C₁₋₄alkyloxycarbonyl, mono- or di(C₁₋₄alkyl)amino, mono- or di(C₁₋₄alkyl)aminoC₁₋₄alkyl, aryl;

r and s are each independently 0, 1, 2, 3, 4 or 5;

t is 0, 1, 2 or 3;

each R¹ and R² are independently hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkylthio, C₁₋₆alkyloxyC₁₋₆alkyloxy, C₁₋₆alkyloxycarbonyl, aminoC₁₋₆alkyloxy, mono- or di(C₁₋₆alkyl)amino, mono- or di(C₁₋₆alkyl)aminoC₁₋₆alkyloxy, aryl, arylC₁₋₆alkyl, aryloxy or arylC₁₋₆alkyloxy, hydroxycarbonyl, C₁₋₆alkyloxycarbonyl, aminocarbonyl, aminoC₁₋₆alkyl, mono- or di(C₁₋₆alkyl)aminocarbonyl, mono- or di(C₁₋₆alkyl)aminoC₁₋₆alkyl; or

two R¹ or R² substituents adjacent to one another on the phenyl ring may independently form together a bivalent radical of formula

| | |
|---|---|
| —O—CH₂—O— | (a-1), |
| —O—CH₂—CH₂—O— | (a-2), |
| —O═CH═CH— | (a-3), |
| —O—CH₂—CH₂— | (a-4), |
| —O—CH₂—CH₂—CH₂— | (a-5), or |
| —CH═CH—CH═CH— | (a-6); |

R³ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, hydroxycarbonyl, hydroxycarbonylC₁₋₆alkyl, C₁₋₆alkyloxycarbonylC₁₋₆alkyl, C₁₋₆alkylcarbonylC₁₋₆alkyl, C₁₋₆alkyloxycarbonyl, aryl, arylC₁₋₆alkyloxyC₁₋₆alkyl, mono- or di(C₁₋₆alkyl)aminoC₁₋₆alkyl;

or a radical of formula

| | |
|---|---|
| —O—R¹⁰ | (b-1), |
| —S—R¹⁰ | (b-2), |
| —NR¹¹R¹² | (b-3), |

wherein

R¹⁰ is hydrogen, C₁₋₆alkyl, C₁₋₆alkylcarbonyl, aryl, arylC₁₋₆alkyl, C₁₋₆alkyloxycarbonylC₁₋₆alkyl, or a radical of formula -Alk-OR¹³ or -Alk-NR¹⁴R¹⁵;

R¹¹ is hydrogen, C₁₋₆alkyl, aryl or arylC₁₋₆alkyl;

R¹² is hydrogen, C₁₋₆alkyl, aryl, hydroxy, amino, C₁₋₆alkyloxy, C₁₋₆alkylcarbonylC₁₋₆alkyl, arylC₁₋₆alkyl, C₁₋₆alkylcarbonylamino, mono- or di(C₁₋₆alkyl)amino, C₁₋₆alkylcarbonyl, aminocarbonyl, arylcarbonyl, haloC₁₋₆alkylcarbonyl, arylC₁₋₆alkylcarbonyl, C₁₋₆alkyloxycarbonyl, C₁₋₆alkyloxyC₁₋₆alkylcarbonyl, mono- or di(C₁₋₆alkyl)aminocarbonyl wherein the alkyl moiety may optionally be substituted by one or more substituents independently selected from aryl or C₁₋₃alkyloxycarbonyl, aminocarbonylcarbonyl, mono- or di(C₁₋₆alkyl)aminoC₁₋₆alkylcarbonyl, or a radical or formula -Alk-OR¹³ or -Alk-NR ¹⁴R¹⁵;

wherein Alk is C₁₋₆alkanediyl;

R¹³ is hydrogen, C₁₋₆alkyl, C₁₋₆alkylcarbonyl, hydroxyC₁₋₆alkyl, aryl or arylC₁₋₆alkyl;

R¹⁴ is hydrogen, C₁₋₆alkyl, aryl or arylC₁₋₆alkyl;

R¹⁵ is hydrogen, C₁₋₆alkyl, C₁₋₆alkylcarbonyl, aryl or arylC₁₋₆alkyl;

R⁴ is a radical of formula

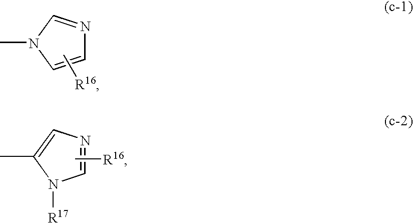

wherein

R¹⁶ is hydrogen, halo, aryl, C₁₋₆alkyl, hydroxyC₁₋₆alkyl, C₁₋₆alkyloxyC₁₋₆alkyl, C₁₋₆alkyloxy, C₁₋₆alkylthio, amino, mono- or di(C₁₋₄alkyl)amino, hydroxycarbonyl, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;

R¹⁶ may also be bound to one of the nitrogen atoms in the imidazole ring of formula (c-1) or (c-2), in which case the meaning of R¹⁶ when bound to the nitrogen is limited to hydrogen, aryl, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl or C₁₋₆alkylS(0)₂C₁₋₆alkyl;

R¹⁷ is hydrogen, C₁₋₆alkyl, C₁₋₆alkyloxyC₁₋₆alkyl, arylC₁₋₆alkyl, trifluoromethyl or di(C₁₋₄alkyl)aminosulfonyl;

R⁵ is C₁₋₆alkyl, C₁₋₆alkyloxy or halo;

aryl is phenyl, naphthalenyl or phenyl substituted with 1 or more substituents each independently selected from halo, C₁₋₆alkyl, C₁₋₆alkyloxy or trifluoromethyl.

The taxane compounds are a class of compounds having the taxane ring system and related to or derived from extracts from certain species of yew (Taxus) trees. These compounds have been found to have activity against tumor cell growth and certain compounds in this class have been used in the clinic for the treatment of various cancers. Thus, for example, paclitaxel is a diterpene isolated from the bark of the the yew tree, Taxus brevifolia, and can be produced by partial synthesis from 10-acetylbacctin, a precursor obtained from yew needles and twigs or by total synthesis, see Holton et al, J. Am. Chem. Soc. 116; 1597-1601 (1994) and Nicholau et al, Nature 367:630 (1994). Paclitaxel has shown neoplastic activity and more recently it has been established that its antitumor activity is due to the promotion of microtubule polymerisation, Kumar N. J., Biol. Chem. 256: 1035-1041 (1981); Rowinsky et al. J. Natl. Cancer Inst. 82: 1247-1259 (1990); and Schiff et al, Nature 277:655-667 (1979). Paclitaxel has now demonstrated efficacy in several human tumors in clinical trials, McGuire et al, Ann. Int. Med. 111: 273-279 (1989); Holmes et al, J. Natl. Cancer Inst. 83: 1797-1805 (1991); Kohn et al J. Natl. Cancer Inst. 86: 18-24 (1994); and Kohn et al, American Society for Clinical Oncology, 12 (1993). Paclitaxel has for example been used for the treatment of ovarian cancer and also breast cancer. Another taxane compound which has been used in the clinic is docetaxel which has been shown to have particular efficacy in the treatment of advanced breast cancer. Docetaxel has shown a better solubility in excipient systems than paclitaxel, therefore increasing the ease with which it can be handled and used in pharmaceutical compositions.

There is therefore a need to increase the inhibitory efficacy of taxane compounds against tumor growth and also to provide a means for the use of lower dosages of taxane compounds to reduce the potential of adverse toxic side effects to the patient.

Certain investigations on a combination of a farnesyl transferase inhibitor and a taxane compound were reported at the 90^{th} Annual meeting of the American Association for Cancer Research (10-14 April 1999, Philadelphia, USA). In in vitro experiments in human tumor cells, an additive anti-tumor effect was observed with a combination of paclitaxel and the farnesyl transferase inhibitor (+)-6-[amino(4-chlorophenyl)(1-methyl-1H-imidazol-5-yl)methyl]-4-(3-chlorophenyl)-1-methyl-2(1H)-quinolinone. In an in vitro mouse colon cancer (LoVo) model an additive anti-tumor effect was also observed with a combination of paclitaxel and the above farnesyl transferase inhibitor. However, these results are not necessarily predictive of the likely therapeutic effect of such a combination in the clinic

It is an object of the invention to provide a therapeutic combination of a taxane compound and a farnesyl transferase inhibitor of the type described above which has an advantageous inhibitory effect against tumor cell growth, in comparison with the respective effects shown by the individual components of the combination.

According to the invention therefore we provide a combination of a taxane compound and a farnesyl transferase inhibitor

of formula (I), (I), (m), (IV), (V), (VI), (VII), (VIII) or (IX) above, in particular a compound of formula (I), (II) or (III):

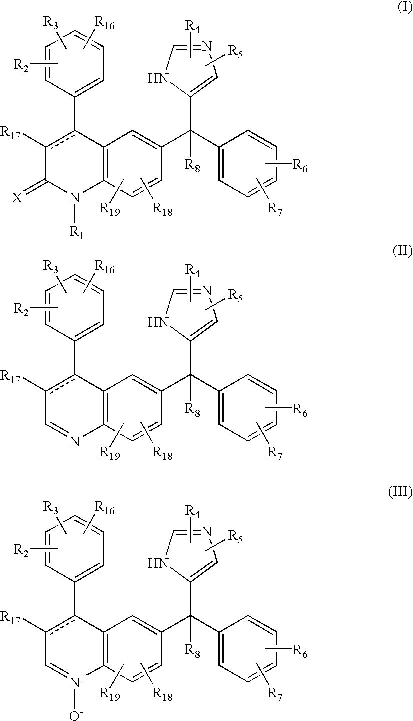

the pharmaceutically acceptable acid or base addition salts and the stereochemically isomeric forms thereof, wherein

the dotted line represents an optional bond;

X is oxygen or sulfur;

R¹ is hydrogen, C₁₋₁₂alkyl, Ar¹, Ar²C₁₋₆alkyl, quinolinylC₁₋₆alkyl, pyridyl-C₁₋₆alkyl, hydroxyC₁₋₆alkyl, C₁₋₆alkyloxyC₁₋₆alkyl, mono- or di (C₁₋₆alkyl)-aminoC₁₋₆alkyl, aminoC₁₋₆alkyl,

or a radical of formula -Alk¹-C(═O)—R⁹, -Alk¹-S(O)—R⁹ or -Alk¹-S(O)₂—R⁹,

wherein Alk¹ is C₁₋₆alkanediyl,

R⁹ is hydroxy, C₁₋₆alkyl, C₁₋₆alkyloxy, amino, C₁₋₈alkylamino or C₁₋₈alkylamino substituted with C₁₋₆alkyloxycarbonyl;

R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or

when on adjacent positions R² and R³ taken together may form a bivalent radical of formula

| | |
|---|---|
| —O—CH₂—O— | (a-1), |
| —O—CH₂—CH₂—O— | (a-2), |
| —O—CH═CH— | (a-3), |
| —O—CH₂—CH₂— | (a-4), |
| —O—CH₂—CH₂—CH₂— | (a-5), or |
| —CH═CH—CH═CH— | (a-6); |

R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)2C₁₋₆alkyl;

R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or

when on adjacent positions R⁶ and R⁷ taken together may form a bivalent radical of formula

| | |
|---|---|
| —O—CH₂—O— | (c-1), or |
| —CH═CH—CH═CH— | (c-2); |

R⁸ is hydrogen, C₁₋₆alkyl, cyano, hydroxycarbonyl, C₁₋₆alkyloxycarbonyl, C₁₋₆alkyl-carbonylC₁₋₆alkyl, cyanoC₁₋₆alkyl, C₁₋₆alkyloxycarbonylC₁₋₆alkyl, carboxy-C₁₋₆alkyl, hydroxyC₁₋₆alkyl, aminoC₁₋₆alkyl, mono- or di(C₁₋₆alkyl)amino-C₁₋₆alkyl, imidazolyl, haloC₁₋₆alkyl, C₁₋₆alkyloxyC₁₋₆alkyl, aminocarbonyl-C₁₋₆alkyl, or a radical of formula

| | |
|---|---|
| —O—R¹⁰ | (b-1), |
| —S—R¹⁰ | (b-2), |
| —N—R¹¹R¹² | (b-3), |

wherein

R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;

R¹¹ is hydrogen, C₁₋₁₂alkyl, Ar¹ or Ar²C₁₋₆alkyl;

R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonyl-C₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₁₆alkylcarbonyl, amino, C₁₋₆alkylamino, C₁₋₆alkylcarbonylamino,

or a radical or formula -Alk²-OR¹³ or -Alk²-NR¹⁴R¹⁵;

wherein Alk² is C₁₋₆alkanediyl;

R¹³ is hydrogen, C₁₋₆alkyl, C₁₋₆alkylcarbonyl, hydroxy-C₁₋₆alkyl, Ar¹ or Ar²C₁₋₆alkyl;

R¹⁴ is hydrogen, C₁₋₆alkyl, Ar¹ or Ar²C₁₋₆alkyl;

R¹⁵ is hydrogen, C₁₋₆alkyl, C₁₋₆alkylcarbonyl, Ar¹ or Ar²C₁₋₆alkyl;

R¹⁷ is hydrogen, halo, cyano, C₁₋₆alkyl, C₁₋₆alkyloxycarbonyl, Ar¹;

R¹⁸ is hydrogen, C₁₋₆alkyl, C₁₋₆alkyloxy or halo;

R¹⁹ is hydrogen or C₁₋₆alkyl;

Ar¹ is phenyl or phenyl substituted with C₁₋₆alkyl, hydroxy, amino, C₁₋₆alkyloxy or halo; and

Ar² is phenyl or phenyl substituted with C₁₋₆alkyl, hydroxy, amino, C₁₋₆alkyloxy or halo.

The above described combinations are hereinafter referred to as combinations according to the invention. These combinations may provide a synergistic effect whereby they demonstrate an advantageous therapeutic effect which is greater than that which would have been expected from the effects of the individual components of the combinations.

In Formulas (I), (II) and (III), R⁴ or R⁵ may also be bound to one of the nitrogen atoms in the imidazole ring. In that case the hydrogen on the nitrogen is replaced by R⁴ or R⁵ and the meaning of R⁴ and R⁵ when bound to the nitrogen is limited to hydrogen, Ar¹, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl, C₁₋₆alkylS(O)2C₁₋₆alkyl.

Preferably the substituent R¹⁸ is situated on the 5 or 7 position of the quinolinone moiety and substituent R¹⁹ is situated on the 8 position when R¹⁸ is on the 7-position.

Interesting compounds are these compounds of formula (I) wherein X is oxygen.

Also interesting compounds are these compounds of formula (I) wherein the dotted line represents a bond, so as to form a double bond.

Another group of interesting compounds are those compounds of formula (I) wherein R¹ is hydrogen, C₁₋₆alkyl, C₁₋₆alkyloxyC₁₋₆alkyl, di(C₁₋₆alkyl)aminoC₁₋₆alkyl, or a radical of formula -Alk¹-C(═O)—R⁹, wherein Alk¹ is methylene and R⁹ is C₁₋₈alkyl-amino substituted with C₁₋₆alkyloxycarbonyl.

Still another group of interesting compounds are those compounds of formula (I) wherein R³ is hydrogen or halo; and R² is halo, C₁₋₆alkyl, C₂₋₆alkenyl, C₁₋₆alkyloxy, trihalomethoxy or hydroxyC₁₋₆alkyloxy.

A further group of interesting compounds are those compounds of formula (I) wherein R² and R³ are on adjacent positions and taken together to form a bivalent radical of formula (a-1), (a-2) or (a-3).

A still further group of interesting compounds are those compounds of formula (I) wherein R⁵ is hydrogen and R⁴ is hydrogen or C₁₋₆alkyl.

Yet another group of interesting compounds are those compounds of formula (1) wherein R⁷ is hydrogen; and R⁶ is C₁₋₆alkyl or halo, preferably chloro, especially 4-chloro.

A particular group of compounds are those compounds of formula (I) wherein R⁸ is hydrogen, hydroxy, haloC₁₋₆alkyl, hydroxyC₁₋₆alkyl, cyanoC₁₋₆alkyl, C₁₋₆alkyloxy-carbonylC₁₋₆alkyl, imidazolyl, or a radical of formula —NR¹¹R¹² wherein R¹¹ is hydrogen or Cl-12alkyl and R¹² is hydrogen, C₁₋₆alkyl, C₁₋₆alkyloxy, hydroxy, C₁₋₆alkyloxyC₁₋₆alkylcarbonyl, or a radical of formula -Alk²-OR¹³ wherein R¹³ is hydrogen or C₁₋₆alkyl.

Preferred compounds are those compounds wherein R¹ is hydrogen, C 1-6alkyl, C₁₋₆alkyloxyC₁₋₆alkyl, di(C₁₋₆alkyl)aminoC₁₋₆alkyl, or a radical of formula -Alk¹-C(═O)—R⁹, wherein Alk¹ is methylene and R⁹ is C₁₋₈alkylamino substituted with C₁₋₆alkyloxycarbonyl; R² is halo, C₁₋₆alkyl, C₂₋₆alkenyl, C₁₋₆alkyloxy, trihalomethoxy, hydroxyC₁₋₆alkyloxy or Ar¹; R³ is hydrogen; R⁴ is methyl bound to the nitrogen in 3-position of the imidazole; R⁵ is hydrogen; R⁶ is chloro; R⁷ is hydrogen; R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, C₁₋₆alkyloxyC₁₋₆alkylcarbonyl, or a radical of formula -Alk²-OR¹³ wherein R¹³ is C₁₋₆alkyl; R¹⁷ is hydrogen and R¹⁸ is hydrogen.

Most preferred compounds are

4-(3-chlorophenyl)-6-[(4-chlorophenyl)hydroxy(1-methyl-1H-imidazol-5-yl)methyl]-1-methyl-2(1H)-quinolinone,

6-[amino(4-chlorophenyl)-1-methyl-1H-imidazol-5-ylmethyl]-4-(3-chlorophenyl)-1-methyl-2(1H)-quinolinone;

6-[(4-chlorophenyl)hydroxy(1-methyl-1H-imidazol-5-yl)methyl]-4-(3-ethoxyphenyl)-1-methyl-2(1H)-quinolinone;

6-[(4-chlorophenyl)(1-methyl-1H-imidazol-5-yl)methyl]-4-(3-ethoxyphenyl)-1-methyl-2(1H)-quinolinone monohydrochloride.monohydrate;

6-[amino(4-chlorophenyl)(1-methyl-1H-imidazol-5-yl)methyl]-4-(3-ethoxyphenyl)-1-methyl-2(1H)-quinolinone,

6-amino(4-chlorophenyl)(1-methyl-1H-imidazol-5-yl)methyl]-1-methyl-4-(3-propylphenyl)-2(1H)-quinolinone; a stereoisomeric form thereof or a pharmaceutically acceptable acid or base addition salt; and

(+)-6-[amino(4-chlorophenyl)(1-methyl-1H-imidazol-5-yl)methyl]-4-(3-chlorophenyl)-1-methyl-2(1H)-quinolinone (Compound 75 in Table 1 of the Experimental part of WO-97/21701) or a pharmaceutically acceptable acid addition salt thereof. The latter compound is especially preferred.

Further preferred embodiments of the present invention include compounds of formula (IX) wherein one or more of the following restrictions apply:

═X¹—X²—X³ is a trivalent radical of formula (x-1), (x-2), (x-3), (x-4) or (x-9) wherein each R⁶ independently is hydrogen, C₁₋₄alkyl, C₁₋₄alkyloxycarbonyl, amino or aryl and R⁷ is hydrogen;

>Y¹—Y²— is a trivalent radical of formula (y-1), (y-2), (y-3), or (y-4) wherein each R⁹ independently is hydrogen, halo, carboxyl, C₁₋₄alkyl or C₁₋₄alkyloxycarbonyl;

r is 0, 1 or 2;

s is 0 or 1;

t is 0;

R¹ is halo, C₁₋₆alkyl or two R¹ substituents ortho to one another on the phenyl ring may independently form together a bivalent radical of formula (a-1);

R² is halo;

R³ is halo or a radical of formula (b-1) or (b-3) wherein

R¹⁰ is hydrogen or a radical of formula -Alk-OR¹³.

R¹¹ is hydrogen;

R¹² is hydrogen, C₁₋₆alkyl, C₁₋₆alkylcarbonyl, hydroxy, C₁₋₆alkyloxy or mono- or di(C₁₋₆alkyl)aminoC₁₋₆alkylcarbonyl;

Alk is C₁₋₆alkanediyl and R¹³ is hydrogen;

R⁴ is a radical of formula (c-1) or (c-2) wherein

R¹⁶ is hydrogen, halo or mono- or di(C₁₋₄alkyl)amino;

R¹⁷ is hydrogen or C₁₋₆alkyl;

aryl is phenyl.

A particular group of compounds consists of those compounds of formula (IX) wherein ═X¹—X²—X³ is a trivalent radical of formula (x-1), (x-2), (x-3), (x-4) or (x-9), >Y1-Y2 is a trivalent radical of formula (y-2), (y-3) or (y4), r is 0 or 1, s is 1, t is 0, R¹ is halo, C₁₋₄)alkyl or forms a bivalent radical of formula (a-1), R² is halo or C₁₋₄alkyl, R³ is hydrogen or a radical of formula (b-1) or (b-3), R⁴ is a radical of formula (c-1) or (c-2), R⁶ is hydrogen, C₁₋₄alkyl or phenyl, R⁷ is hydrogen, R⁹ is hydrogen or C₁₋₄alkyl, R¹⁰ is hydrogen or -Alk-OR¹³, R¹¹ is hydrogen and R¹² is hydrogen or C₁₋₆alkylcarbonyl and R¹³ is hydrogen;

Preferred compounds are those compounds of formula (IX) wherein ═X¹—X²—X³ is a trivalent radical of formula (x-1) or (x-4), >Y1-Y2 is a trivalent radical of formula (y-4), r is 0 or 1, s is 1, t is 0, R¹ is halo, preferably chloro and most preferably 3-chloro, R² is halo, preferably 4-chloro or 4-fluoro, R³ is hydrogen or a radical of formula (b-1) or (b-3), R⁴ is a radical of formula (c-1) or (c-2), R⁶ is hydrogen, R⁷ is hydrogen, R⁹ is hydrogen, R¹⁰ is hydrogen, R¹¹ is hydrogen and R¹² is hydrogen;

Other preferred compounds are those compounds of formula (IX) wherein ═X¹—X²—X³ is a trivalent radical of formula (x-2), (x-3) or (x-4), >Y1-Y2 is a trivalent radical of formula (y-2), (y-3) or (y-⁴), r and s are 1, t is 0, R¹ is halo, preferably chloro, and most preferably 3-chloro or R¹ is C₁₋₄alkyl, preferably 3-methyl, R² is halo, preferably chloro, and most preferably 4-chloro, R³ is a radical of formula (b-1) or (b-3), R⁴ is a radical of formula (c-2), R⁶ is C₁₄alkyl, R⁹ is hydrogen, R¹⁰ and R¹¹ are hydrogen and R¹² is hydrogen or hydroxy.

The most preferred compounds of formula (IX) are

7-[(4-fluorophenyl)(1H-imidazol-1-yl)methyl]-5-phenylimidazo[1,2-a]quinoline;

α-(4-chlorophenyl)-(α-(1-methyl-1H-imidazol-5-yl)-5-phenylimidazo[1,2-a]quinoline-7-methanol;

5-(3-chlorophenyl)-(α-(4-chlorophenyl)-α-(1-methyl-1H-imidazol-5-yl)-imidazo-[1,2-a]quinoline-7-methanol;

5-(3-chlorophenyl)-(α-(4-chlorophenyl)-α-(1-methyl-1H-imidazol-5-yl)imidazo-[1,2-a]quinoline-7-methanamine;

5-(3-chlorophenyl)-α-(4-chlorophenyl)-α-(1-methyl-1H-imidazol-5-yl)tetrazolo-[1,5-a]quinoline-7-methanamine;

5-(3-chlorophenyl)-α-(4-chlorophenyl)-1-methyl-α-(1-methyl-1H-imidazol-5-yl)-1,2,4-triazolo[4,3-a]quinoline-7-methanol;

5-(3-chlorophenyl)-α-(4-chlorophenyl)-α-(1-methyl-1H-imidazol-5-yl)tetrazolo-[1,5-a]quinoline-7-methanamine;

5-(3-chlorophenyl)-α-(4-chlorophenyl)-α-(1-methyl-1H-imidazol-5-yl)tetrazolo-[1,5-a]quinazoline-7-methanol;

5-(3-chlorophenyl)-α-(4-chlorophenyl)-4,5-dihydro-α-(1-methyl-1H-imidazol-5-yl)tetrazolo[1,5-a]quinazoline-7-methanol;

5-(3-chlorophenyl)-α-(4-chlorophenyl)-α-(1-methyl-1H-imidazol-5-yl)tetrazolo-[1,5-a]quinazoline-7-methanamine;

5-(3-chlorophenyl)-α-(4-chlorophenyl)-N-hydroxy-α-(1-methyl-1H-imidazol-5-yl)-tetrahydro[1,5-a]quinoline-7-methanamine;

α-(4-chlorophenyl)-α-(1-methyl-1H-imidazol-5-yl)-5-(3-methylphenyl)tetrazolo-[1,5-a]quinoline-7-methanamine; the pharmaceutically acceptable acid addition salts and the stereochemically isomeric forms thereof.

5-(3-chlorophenyl)-α-(4-chlorophenyl)-α-(1-methyl-1H-imidazol-5-yl)tetrazolo-[1,5-a]quinazoline-7-methanamine, especially the (−) enantiomer, and its pharmaceutically acceptable acid addition salts are especially preferred.

As used in the foregoing definitions and hereinafter halo defines fluoro, chloro, bromo and iodo; C₁₋₆alkyl defines straight and branched chained saturated hydrocarbon radicals having from 1 to 6 carbon atoms such as, for example, methyl, ethyl, propyl, butyl, pentyl, hexyl and the like; C₁₋₈alkyl encompasses the straight and branched chained saturated hydrocarbon radicals as defined in C₁₋₆alkyl as well as the higher homologues thereof containing 7 or 8 carbon atoms such as, for example heptyl or octyl; C₁₋₂alkyl again encompasses C₁₋₈alkyl and the higher homologues thereof containing 9 to 12 carbon atoms, such as, for example, nonyl, decyl, undecyl, dodecyl; C₁₋₁₆alkyl again encompasses C₁₋₁₂alkyl and the higher homologues thereof containing 13 to 16 carbon atoms, such as, for example, tridecyl, tetradecyl, pentedecyl and hexadecyl; C₂₋₆alkenyl defines straight and branched chain hydrocarbon radicals containing one double bond and having from 2 to 6 carbon atoms such as, for example, ethenyl, 2-propenyl, 3-butenyl, 2-pentenyl, 3-pentenyl, 3-methyl-2-butenyl, and the like; C₁₋₆alkanediyl defines bivalent straight and branched chained saturated hydrocarbon radicals having from 1 to 6 carbon atoms, such as, for example, methylene, 1,2-ethanediyl, 1,3-propanediyl, 1,4-butanediyl, 1,5-pentanediyl, 1,6-hexanediyl and the branched isomers thereof. The term “C(═O)” refers to a carbonyl group, “S(O)” refers to a sulfoxide and “S(O)2” to a sulfon. The term “natural amino acid” refers to a natural amino acid that is bound via a covalent amide linkage formed by loss of a molecule of water between the carboxyl group of the amino acid and the amino group of the remainder of the molecule. Examples of natural amino acids are glycine, alanine, valine, leucine, isoleucine, methionine, proline, phenylanaline, tryptophan, serine, threonine, cysteine, tyrosine, asparagine, glutamine, aspartic acid, glutamic acid, lysine, arginine, histidine.

The pharmaceutically acceptable acid or base addition salts as mentioned hereinabove are meant to comprise the therapeutically active non-toxic acid and non-toxic base addition salt forms which the compounds of formulas (I), (II), (III), (IV), (V), (VI), (VII), (VIII) or (IX) are able to form. The compounds of formulas (I), (II), (III), (IV), (V), (VI), (VII), (VIII) or (IX) which have basic properties can be converted in their pharmaceutically acceptable acid addition salts by treating said base form with an appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedloic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The compounds of formulae (I), (II), (III), (IV), (V), (VI), (VII), (VIII) or (IX) which have acidic properties may be converted in their pharmaceutically acceptable base addition salts by treating said acid form with a suitable organic or inorganic base. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The terms acid or base addition salt also comprise the hydrates and the solvent addition forms which the compounds of formulae (I), (II), (III), (IV), (V), (VI), (VII), (VIII) or (IX) are able to form. Examples of such forms are e.g. hydrates, alcoholates and the like.

The term stereochemically isomeric forms of compounds of formulae (I), (II), (III), (IV), (V), (VI), (VII), (VII) or (IX), as used hereinbefore, defines all possible compounds made up of the same atoms bonded by the same sequence of bonds but having different three-dimensional structures which are not interchangeable, which the compounds of formulae (I), (II), (III), (IV), (V), (VI), (VII), (VIII) or (IX) may possess. Unless otherwise mentioned or indicated, the chemical designation of a compound encompasses the mixture of all possible stereochemically isomeric forms which said compound may possess. Said mixture may contain all

diastereomers and/or enantiomers of the basic molecular structure of said compound. All stereochemically isomeric forms of the compounds of formulae (I), (II), (III), (IV), (V), (VI), (VII), (VIII) or (IX) both in pure form or in admixture with each other are intended to be embraced within the scope of the present invention.

Some of the compounds of formulae (I), (II), (III), (IV), (V), (VI), (VII), (VIII) or (IX) may also exist in their tautomeric forms. Such forms although not explicitly indicated in the above formula are intended to be included within the scope of the present invention.

Whenever used hereinafter, the term “compounds of formulae (I), (II), (III), (IV), (V), (VI), (VII), (VIII) or (IX)” is meant to include also the pharmaceutically acceptable acid or base addition salts and all stereoisomeric forms.

The taxane compound used in the combinations according to the invention is preferably paclitaxel or docetaxel referred to above. Paclitaxel is available commercially for example under the trade name Taxol from Bristol Myers Squibb and docetaxel is available commercially under the trade name Taxotere from Rhone-Poulenc Rorer. Both compounds and other taxane compounds may be prepared in conventional manner for example as described in EP 253738, EP 253739 and WO 92/09589 or by processes analogous thereto.

The present invention also relates to combinations according to the invention for use in medical therapy for example for inhibiting the growth of tumor cells.

The present invention also relates to the use of combinations according to the invention for the preparation of a pharmaceutical composition for inhibiting the growth of tumor cells.

The present invention also relates to a method of inhibiting the growth of tumor cells in a human subject which comprises administering to the subject an effective amount of a combination according to the invention.

This invention further provides a method for inhibiting the abnormal growth of cells, including transformed cells, by administering an effective amount of a combination according to the invention. Abnormal growth of cells refers to cell growth independent of normal regulatory mechanisms (e.g. loss of contact inhibition). This includes the abnormal growth of: (1) tumor cells (tumors) expressing an activated ras oncogene; (2) tumor cells in which the ras protein is activated as a result of oncogenic mutation of another gene; (3) benign and malignant cells of other proliferative diseases in which aberrant ras activation occurs. Furthermore, it has been suggested in literature that ras oncogenes not only contribute to the growth of of tumors in vivo by a direct effect on tumor cell growth but also indirectly, i.e. by facilitating tumor-induced angiogenesis (Rak. J. et al, Cancer Research, 55, 4575-4580, 1995). Hence, pharmacologically targetting mutant ras oncogenes could conceivably suppress solid tumor growth in vivo, in part, by inhibiting tumor-induced angiogenesis.

This invention also provides a method for inhibiting tumor growth by administering an effective amount of a combination according to the present invention, to a subject, e.g. a mammal (and more particularly a human) in need of such treatment. In particular, this invention provides a method for inhibiting the growth of tumors expressing an activated ras oncogene by the administration of an effective amount of combination according to the present invention. Examples of tumors which may be inhibited include, but are not limited to, lung cancer (e.g. adenocarcinoma and including non-small cell lung cancer), pancreatic cancers (e.g. pancreatic carcinoma such as, for example exocrine pancreatic carcinoma), colon cancers (e.g. colorectal carcinomas, such as, for example, colon adenocarcinoma and colon adenoma), hematopoietic tumors of lymphoid lineage (e.g. acute lymphocytic leukemia, B-cell lymphoma, Burkitt's lymphoma), myeloid leukemias (for example, acute myelogenous leukemia (AML)), thyroid follicular cancer, myelodysplastic syndrome (MDS), tumors of mesenchymal origin (e.g. fibrosarcomas and rhabdomyosarcomas), melanomas, teratocarcinomas, neuroblastomas, gliomas, benign tumor of the skin (e.g. keratoacanthomas), breast carcinoma (e.g. advanced breast cancer), kidney carninoma, ovary carcinoma, bladder carcinoma and epidermal carcinoma.

This invention also provides a method for inhibiting proliferative diseases, both benign and malignant, wherein ras proteins are aberrantly activated as a result of oncogenic mutation in genes, i.e. the ras gene itself is not activated by mutation to an oncogenic mutation to an oncogenic form, with said inhibition being accomplished by the administration of an effective amount of a combination according to the invention, to a subject in need of such a treatment. For example, the benign proliferative disorder neurofibromatosis, or tumors in which ras is activated due to mutation or overexpression of tyrosine kinase oncogenes may be inhibited by the combinations according to the invention.

The taxane compound and the farnesyl transferase inhibitor may be administered simultaneously (e.g. in separate or unitary compositions) or sequentially in either order. In the latter case, the two compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular taxane compound and farnesyl transferase inhibitor being administered, their route of administration, the particular tumor being treated and the particular host being treated. The optimum method and order of administration and the dosage amounts and regime can be readily determined by those skilled in the art using conventional methods and in view of the information set out herein.

The farnesyl transferase inhibitor is advantageously administered in an effective amount of from 0.0001 mg/kg to 100 mg/kg body weight, and in particular from 0.001 mg/kg to 10 mg/kg body weight. More particularly, for an adult patient, the dosage is conveniently in the range of 50 to 500 mg bid, advantageously 100 to 400 mg bid and particularly 300 mg bid.

The taxane compound is advantageously administered in a dosage of 50 to 400 mg per square meter (mg/m²) of body surface area, for example 75 to 250 mg/m², particularly for paclitaxel in a dosage of about 175 to 250 mg/m² and for docetaxel in about 75 to 150 mg/m² per course of treatment. These dosages may be administered for example once, twice or more per course of treatment, which may be repeated for example every 7, 14, 21 or 28 days.

It is especially preferred to administer the farnesyl tranferase inhibitor at a dosage of 100 or 200 mg bid for 7, 14, 21 or 28 days with a dosage of the taxane compound in the ranges indicated above.

In view of their useful pharmacological properties, the components of the combinations according to the invention, i.e. the taxane compound and the farnesyl transferase inhibitor may be formulated into various pharmaceutical forms for administration purposes. The components may formulated separately in individual pharmaceutical compositions or in a unitary pharmaceutical composition containing both components. Farnesyl protein transferase inhibitors can be prepared and formulated into pharmaceutical compositions by methods known in the art and in particular according to the methods described in the published patent specifications mentioned herein and incorporated by reference; for the compounds of formulae (I), (II) and (III) suitable examples can be found in WO-97/21701. Compounds of formulae (IV), (V), and (VI) can be prepared and formulated using methods described in WO 97/16443, compounds of formulae (VII) and (VIII) according to methods described in WO 98/40383 and WO 98/49157 and compounds of formula (IX) according to methods described in WO 00/39082 respectively.

The present invention therefore also relates to a pharmaceutical composition comprising a taxane compound and a farnesyl tranferase inhibitor of formula (I) together with one or more pharmaceutical carriers. To prepare pharmaceutical compositions for use in accordance with the invention, an effective amount of a particular compound, in base or acid addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, preferably, for administration orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, to aid solubility for example, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect to the skin. Said additives may facilitate the administration to the skin and/or may be helpful for preparing the desired compositions. These compositions may be administered in various ways, e.g., as a transdermal patch, as a spot-on, as an ointment. It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

It may be appropriate to administer the required dose of each component of the combination as two, three, four or more sub-doses at appropriate intervals throughout the course of treatment Said sub-doses may be formulated as unit dosage forms, for example, in each case containing independently 0.01 to 500 mg, for example 0.1 to 200 mg and in particular 1 to 100 mg of each active ingredient per unit dosage form.

Experimental Testing of Combinations for Inhibition of Tumor Growth

The combinations according to the invention may be tested for their efficacy in inhibiting tumor growth using conventional assays described in the literature for example the HTB177 lung carcinoma described by Liu M et al, Cancer Research, Vol. 58, No.21, 1 November 1998, pages 4947-4956, and the anti-mitotic assay described by Moasser M et al, Proc. Natl. Acad. Sci. USA, Vol. 95, pages 1369-1374, February 1998. Other in vitro and in vivo models for determining ant-tumor effects of combinations and possible synergy of the combinations according to the invention are described in WO 98/54966 and WO 98/32114. Clinical models for determining the efficacy and possible synergism for combination therapy in the clinic are generally described in Cancer: Principles and Practice of Oncology, Fifth Edition, edited by Vincent T DeVita, Jr., Samuel Hellman, Steven A. Rosenberg, Lippincott-Raven, Philadelphia, 1997, especially Chapter 17, pages 342-346.

1. A combination of a taxane compound and a farnesyl transferase inhibitor selected from compounds of formulae (I), (II), (III), (IV), (V), (VI), (VII), (VIII) and (IX) below: 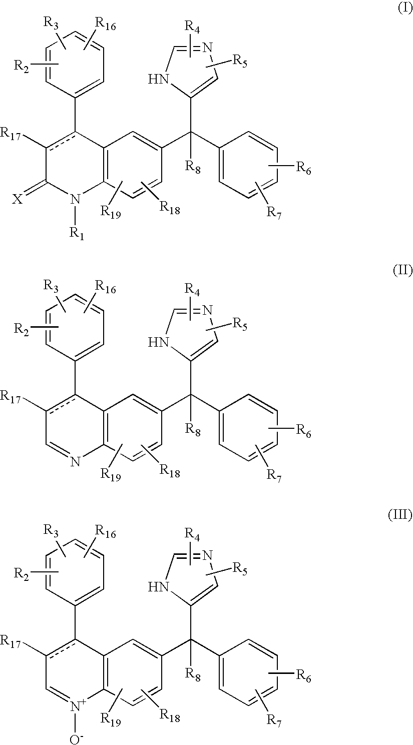
the pharmaceutically acceptable acid or base addition salts and the stereochemically isomeric forms thereof, wherein
the dotted line represents an optional bond;
X is oxygen or sulfur;
R¹ is hydrogen, C₁₋₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl,
or a radical of formula -Alk¹-C(═O)—R⁹, -Alk¹-S(O)—R⁹ or -Alk¹-S(O)₂—R⁹,
wherein Alk¹ is C₁₋₆alkanediyl,
R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyl-oxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or
when on adjacent positions R² and R³ taken together may form a bivalent radical of formula

| | |
|---|---|
| —O—CH₂—O— | (a-1), |
| —O—CH₂—CH₂—O— | (a-2), |
| —O—CH═CH— | (a-3), |
| —O—CH₂—CH₂— | (a-4), |
| —O—CH₂—CH₂—CH₂— | (a-5), or |
| —CH═CH—CH═CH— | (a-6); |

R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or
when on adjacent positions R⁶ and R⁷ taken together may form a bivalent radical of formula

| | |
|---|---|
| —O—CH₂—O— | (c-1), or |
| —CH═CH—CH═CH— | (c-2); |

R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)-aminoC₁₋₆alkyl, imidazolyl, haloC₁₋₆alkyl, C₁₋₆alkyloxyC₁₋₆alkyl, aminocarbonylC₁₋₆alkyl, or a radical of formula

| | |
|---|---|
| —O—R¹⁰ | (b-1), |
| —S—R¹⁰ | (b-2), |
| —N—R¹¹R¹² | (b-3), |

wherein
R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;
R¹¹ is hydrogen, C₁₋₁₂alkyl, Ar¹ or Ar²C₁₋₆alkyl;
R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonyl-C₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl amino, C₁₋₆alkylamino, C₁₋₆alkylcarbonylamino, or a radical or formula -Alk²-OR ¹³ or -Alk²-NR ¹⁴R¹⁵;
wherein Alk² is C₁₋₆alkanediyl;
R¹³ is hydrogen, C₁₋₆alkyl, C₁₋₆alkylcarbonyl, hydroxy-C₁₋₆alkyl, Ar¹ or Ar²C₁₋₆alkyl;
R¹⁴ is hydrogen, C₁₋₆alkyl, Ar¹ or Ar²C₁₋₆alkyl;
R¹⁵ is hydrogen, C₁₋₆alkyl, C₁₋₆alkylcarbonyl, Ar¹ or Ar²C₁₋₆alkyl;
R¹⁷ is hydrogen, halo, cyano, C₁₋₆alkyl, C₁₋₆alkyloxycarbonyl, Ar¹;
R¹⁸ is hydrogen, C₁₋₆alkyl, C₁₋₆alkyloxy or halo;
R¹⁹ is hydrogen or C₁₋₆alkyl;
Ar¹ is phenyl or phenyl substituted with C₁₋₆alkyl, hydroxy, amino, C₁₋₆alkyloxy or halo; and
Ar² is phenyl or phenyl substituted with C₁₋₆alkyl, hydroxy, amino, C₁₋₆alkyloxy or halo. 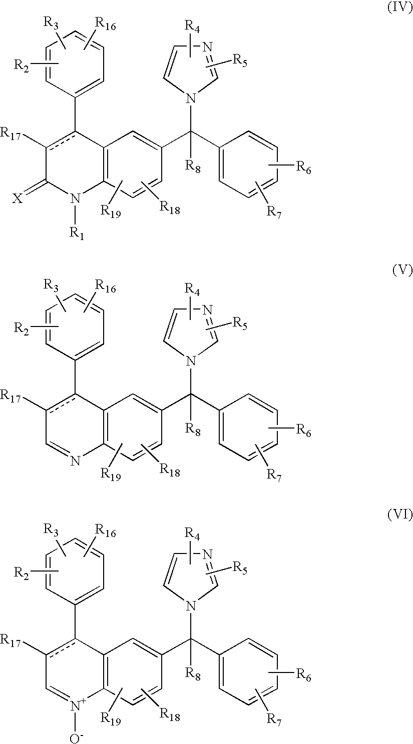
the pharmaceutically acceptable acid or base addition salts and the stereochemically isomeric forms thereof, wherein
the dotted line represents an optional bond;
X is oxygen or sulfur;
R¹ is hydrogen, C₁₋₂alkyl, Ar¹, Ar²C₁₋₆alkyl, quinolinylC₁₋₆alkyl, pyridyl-C₁₋₆alkyl, hydroxyC₁₋₆alkyl, C₁₋₆alkyloxyC₁₋₆alkyl, mono- or di(C₁₋₆alkyl)-aminoC₁₋₆alkyl, aminoC₁₋₆alkyl,
or a radical of formula -Alk¹-C(═O)—R⁹, -Alk¹-S(O)-R⁹ or -Alk¹-S(O)₂—R⁹,
wherein Alk¹ is C₁₋₆alkanediyl,
R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R² and R³ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, amino-C₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl; or
when on adjacent positions R² and R³ taken together may form a bivalent radical of formula

| | |
|---|---|
| —O—CH₂—O— | (a-1), |
| —O—CH₂—CH₂—O— | (a-2), |
| —O—CH═CH— | (a-3), |
| —O—CH₂—CH₂— | (a-4), |
| —O—CH₂—CH₂—CH₂— | (a-5), or |
| —CH═CH—CH═CH— | (a-6); |

R⁴ and R⁵ each independently are hydrogen, Ar¹, C₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy or Ar²oxy;
R⁸ is hydrogen, C₁₋₆alkyl, cyano, hydroxycarbonyl, C₁₋₆alkyloxycarbonyl, C₁₋₆alkyl-carbonylC₁₋₆alkyl, cyanoC₁₋₆alkyl, C₁₋₆alkyloxycarbonylC₁₋₆alkyl, hydroxy-carbonylC₁₋₆alkyl, hydroxyC₁₋₆alkyl, aminoC₁₋₆alkyl, mono- or di(C₁₋₆alkyl)-aminoC₁₋₆alkyl, haloC₁₋₆alkyl, C₁₋₆alkyloxyC₁₋₆alkyl, aminocarbonylC₁₋₆alkyl, Ar¹, Ar²C₁₋₆alkyloxyC₁₋₆alkyl, C₁₋₆alkylthioC₁₋₆alkyl;
R¹⁰ is hydrogen, C₁₋₆alkyl, C₁₋₆alkyloxy or halo;
R¹¹ is hydrogen or C₁₋₆alkyl;
Ar¹ is phenyl or phenyl substituted with C₁₋₆alkyl, hydroxy, amino, C₁₋₆alkyloxy or halo;
Ar² is phenyl or phenyl substituted with C₁₋₆alkyl, hydroxy, amino, C₁₋₆alkyloxy or halo. 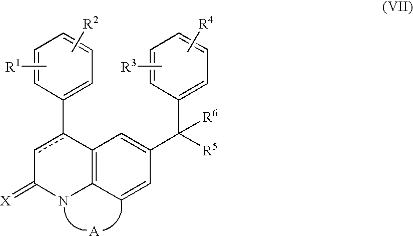
the pharmaceutically acceptable acid addition salts and the stereochemically isomeric forms thereof, wherein
the dotted line represents an optional bond;
X is oxygen or sulfur;
-A- is a bivalent radical of formula

| | |
|---|---|
| —CH═CH— | (a-1), |
| —CH₂—CH₂— | (a-2), |
| —CH₂—CH₂—CH₂— | (a-3), |
| —CH₂—O— | (a-4), |
| —CH₂—CH₂—O— | (a-5), |
| —CH₂—S— | (a-6), |
| —CH₂—CH₂—S— | (a-7), |
| —CH═N— | (a-8), |
| —N═N— | (a-9), or |
| —CO—NH— | (a-10); |

wherein optionally one hydrogen atom may be replaced by C14alkyl or Ar¹;
R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar², Ar²-C₁₋₆alkyl, Ar²-oxy, Ar²-C₁₋₆alkyloxy; or when on adjacent positions R¹ and R² taken together may form a bivalent radical of formula

| | |
|---|---|
| —O—CH₂—O— | (b-1), |
| —O—CH₂—CH₂—O— | (b-2), |
| —O-CH═CH— | (b-3), |
| —O—CH₂—CH₂— | (b-4), |
| —O—CH₂—CH₂—CH₂— | (b-5), or |
| —CH═CH—CH═CH— | (b-6); |

R³ and R⁴ each independently are hydrogen, halo, cyano, C₁₋₆alkyl, C₁₋₆alkyloxy, Ar³-oxy, C₁₋₆alkylthio, di(C₁₋₆alkyl)amino, trihalomethyl, trihalomethoxy, or when on adjacent positions R³ and R⁴ taken together may form a bivalent radical of formula

| | |
|---|---|
| —O—CH₂—O— | (c-1), |
| —O—CH₂—CH₂—O— | (c-2), or |
| —CH═CH—CH═CH— | (c-3); |

R⁵ is a radical of formula 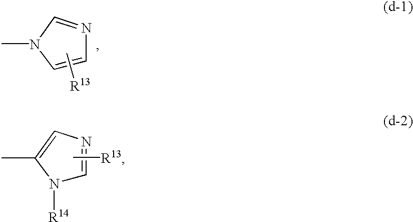
wherein
R¹³ is hydrogen, halo, Ar⁴, C₁₋₆alkyl, hydroxyC₁₋₆alkyl, C₁₋₆alkyloxy-C₁₋₆alkyl, C₁₋₆alkyloxy, C₁₋₆alkylthio, amino, C₁₋₆alkyloxy-carbonyl, C₁₋₆alkylS(O)C₁₋₆alkyl or C₁₋₆alkylS(O)₂C₁₋₆alkyl;
R¹⁴ is hydrogen, C₁₋₆alkyl or di(C₁₋₄alkyl)aminosulfonyl;
R⁶ is hydrogen, hydroxy, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar⁵, Ar⁵-C₁₋₆alkyloxyC₁₋₆alkyl; or a radical of formula
—O—R⁷ (e-1),
—S—R⁷ (e-2),
—N—R⁸R⁹ (e-3),
wherein
R⁷ is hydrogen, C₁₋₆alkyl, C₁₋₆alkylcarbonyl, Ar⁶, Ar⁶-C₁₋₆alkyl, C₁₋₆alkyloxycarbonylC₁₋₆alkyl, or a radical of formula -Alk-OR¹⁰ or -Alk-NR¹¹R¹²;
R⁸ is hydrogen, C₁₋₆alkyl, Ar⁷ or Ar⁷-C₁₋₆alkyl;
R⁹ is hydrogen, C₁₋₆alkyl, C₁₋₆alkylcarbonyl, C₁₋₆alkyloxycarbonyl, C₁₋₆alkylaminocarbonyl, Ar⁸, Ar⁸-C₁₋₆alkyl, C₁₋₆alkylcarbonyl-C₁₋₆alkyl, Ar⁸-carbonyl, Ar⁸-C₁₋₆alkylcarbonyl, aminocarbonyl-carbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino,
or a radical or formula -Alk-OR¹⁰ or -Alk-NR¹¹R¹²;
wherein Alk is C₁₋₆alkanediyl;
R¹⁰ is hydrogen, C₁₋₆alkyl, C₁₋₆alkylcarbonyl, hydroxyC₁₋₆alkyl, Ar⁹ or Ar⁹-C₁₋₆alkyl;
R¹¹ is hydrogen, C₁₋₆alkyl, C₁₋₆alkylcarbonyl, Ar¹⁰ or Ar¹⁰-C₁₋₆alkyl;
R¹² is hydrogen, C₁₋₆alkyl, Ar¹¹ or Ar¹¹-C₁₋₆alkyl; and
Ar¹ to Ar¹¹ are each independently selected from phenyl; or phenyl substituted with halo, C₁₋₆alkyl, C₁₋₆alkyloxy or trifluoromethyl. 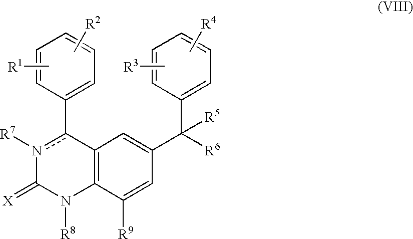
the pharmaceutically acceptable acid addition salts and the stereochermically isomeric forms thereof, wherein
the dotted line represents an optional bond;
X is oxygen or sulfur;
R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar¹, Ar¹C₁₋₆alkyl, Ar¹oxy or
Ar¹C₁₋₆alkyloxy;
R³ and R⁴ each independently are hydrogen, halo, cyano, C₁₋₆alkyl, C₁₋₆alkyloxy, Ar¹oxy, C₁₋₆alkylthio, di(C₁₋₆alkyl)amino, trihalomethyl or trihalomethoxy;
R⁵ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar¹, Ar¹C₁₋₆alkyloxyC₁₋₆alkyl; or a radical of formula

| | |
|---|---|
| —O—R¹⁰ | (a-1), |
| —S—R¹⁰ | (a-2), |
| —N—R¹¹R¹² | (a-3), |

wherein
R¹⁰ is hydrogen, C₁₋₆alkyl, C₁₋₆alkylcarbonyl, Ar¹, Ar¹C₁₋₆alkyl, C₁₋₆alkyloxycarbonylC₁₋₆alkyl, or a radical of formula -Alk-OR¹³ or -Alk-NR¹⁴R¹⁵;
R¹¹ is hydrogen, C₁₋₆alkyl, Ar¹ or Ar¹C₁₋₆alkyl;
R¹² is hydrogen, C₁₋₆alkyl, C₁₋₆alkylcarbonyl, C₁₋₆alkyloxycarbonyl, C₁₋₆alkylaminocarbonyl, Ar¹, Ar¹C₁₋₆alkyl, C₁₋₆alkylcarbonyl-C₁₋₆alkyl, Ar¹carbonyl, Ar¹C₁₋₆alkylcarbonyl, aminocarbonyl-carbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino,
or a radical or formula -Alk-OR¹³ or -Alk-NR¹⁴R¹⁵;
wherein Alk is C₁₋₆alkanediyl;
R¹³ is hydrogen, C₁₋₆alkyl, C₁₋₆alkylcarbonyl, hydroxy-C₁₋₆alkyl, Ar¹ or Ar¹C₁₋₆alkyl;
R¹⁴ is hydrogen, C₁₋₆alkyl, Ar¹ or Ar¹C₁₋₆alkyl;
R¹⁵ is hydrogen, C₁₋₆alkyl, C₁₋₆alkylcarbonyl, Ar¹ or Ar¹C₁₋₆alkyl;
R⁶ is a radical of formula 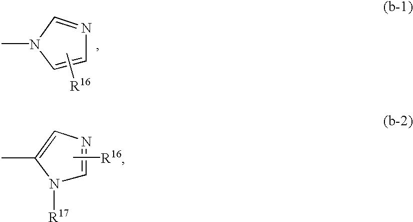
wherein
R¹⁶ is hydrogen, halo, Ar¹, C₁₋₆alkyl, hydroxyC₁₋₆alkyl, C₁₋₆alkyloxy-C₁₋₆alkyl, C₁₋₆alkyloxy, C₁₋₆alkylthio, amino, C₁₋₆alkyloxycarbonyl, C₁₋₆alkylthioC₁₋₆alkyl, C₁₋₆alkylS(O)C₁₋₆alkyl or C₁₋₆alkylS(O)2C₁₋₆alkyl;
R¹⁷ is hydrogen, C₁₋₆alkyl or di(C₁₋₄alkyl)aminosulfonyl;
R⁷ is hydrogen or C₁₋₆alkyl provided that the dotted line does not represent a bond;
R⁸ is hydrogen, C₁₋₆alkyl or Ar²CH₂ or Het¹CH₂;
R⁹ is hydrogen, C₁₋₆alkyl, C₁₋₆alkyloxy or halo; or
R⁸ and R⁹ taken together to form a bivalent radical of formula

| | |
|---|---|
| —CH═CH— | (c-1), |
| —CH₂—CH₂— | (c-2), |
| —CH₂—CH₂—CH₂— | (c-3), |
| —CH₂—O— | (c-4), or |
| —CH₂—CH₂—O— | (c-5); |

Ar¹ is phenyl; or phenyl substituted with 1 or 2 substituents each independently selected from halo, C₁₋₆alkyl, C₁₋₆alkyloxy or trifluoromethyl;
Ar² is phenyl; or phenyl substituted with 1 or 2 substituents each independently selected from halo, C₁₋₆alkyl, C₁₋₆alkyloxy or trifluoromethyl; and
Het¹ is pyridinyl; pyridinyl substituted with 1 or 2 substituents each independently selected from halo, C₁₋₆alkyl, C₁₋₆alkyloxy or trifluoromethyl and 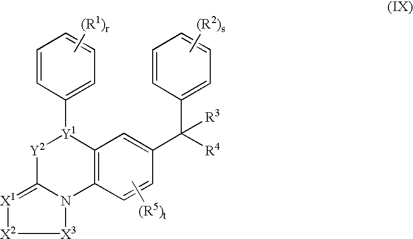
or the pharmaceutically acceptable acid addition salts and the stereochemically isomeric forms thereof, wherein
═X¹—X²—X³— is a trivalent radical of formula

| | |
|---|---|
| ═N—CR⁶═CR⁷— | (x-1), |
| ═N—N═CR⁶— | (x-2), |
| ═N—NH—C(═O)— | (x-3), |
| ═N—N═N— | (x-4), |
| ═N—CR⁶═N— | (x-5), |
| ═CR⁶—CR⁷═CR⁸— | (x-6), |
| ═CR⁶—N═CR⁷— | (x-7), |
| ═CR⁶—NH—C(═O)— | (x-8), or |
| ═CR⁶—N═N— | (x-9); |

wherein each R⁶, R⁷ and R⁸ are independently hydrogen, C₁₋₄alkyl, hydroxy, C₁₋₄alkyloxy, aryloxy, C₁₋₄alkyloxycarbonyl, hydroxyC₁₋₄alkyl, C₁₋₄alkyloxyC₁₋₄alkyl, mono- or di(C₁₋₄alkyl)aminoC₁₋₄alkyl, cyano, amino, thio, C₁₋₄alkylthio, arylthio or aryl;
>Y¹—Y²— is a trivalent radical of formula

| | |
|---|---|
| >CH—CHR⁹— | (y-1), |
| >C═N— | (y-2), |
| >CH—NR⁹— | (y-3),or |
| >C═CR⁹— | (y-4); |

wherein each R⁹ independently is hydrogen, halo, halocarbonyl, aminocarbonyl, hydroxyC₁₋₄alkyl, cyano, carboxyl, C₁₋₄alkyl, C₁₋₄alkyloxy, C₁₋₄alkyloxyC₁₋₄alkyl, C₁₋₄alkyloxycarbonyl, mono- or di(C₁₋₄alkyl)amino, mono- or di(C₁₋₄alkyl)aminoC₁₋₄alkyl, aryl;
r and s are each independently 0, 1, 2, 3, 4 or 5;
t is 0, 1, 2 or 3;
each R¹ and R² are independently hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkylthio, C₁₋₆alkyloxyC₁₋₆alkyloxy, C₁₋₆alkyloxycarbonyl, aminoC₁₋₆alkyloxy, mono- or di(C₁₋₆alkyl)amino, mono- or di(C₁₋₆alkyl)aminoC₁₋₆alkyloxy, aryl, arylC₁₋₆alkyl, aryloxy or arylC₁₋₆alkyloxy, hydroxycarbonyl, C₁₋₆alkyloxycarbonyl, aminocarbonyl, aminoC₁₋₆alkyl, mono- or di(C₁₋₆alkyl)aminocarbonyl, mono- or di(C₁₋₆alkyl)aminoC₁₋₆alkyl; or
two R¹ or R² substituents adjacent to one another on the phenyl ring may independently form together a bivalent radical of formula

| | |
|---|---|
| —O—CH₂—O— | (a-1), |
| —O—CH₂—CH₂—O— | (a-2), |
| —O═CH₂═CH— | (a-3), |
| —O—CH₂—CH₂— | (a-4), |
| —O—CH₂—CH₂—CH₂— | (a-5), or |
| —CH═CH—CH═CH— | (a-6); |

R³ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, hydroxycarbonyl, hydroxycarbonylC₁₋₆alkyl, C₁₋₆alkyloxycarbonylC₁₋₆alkyl, C₁₋₆alkylcarbonylC₁₋₆alkyl, C₁₋₆alkyloxycarbonyl, aryl, arylC₁₋₆alkyloxyC₁₋₆alkyl, mono- or di(C₁₋₆alkyl)aminoC₁₋₆alkyl;
or a radical of formula

| | |
|---|---|
| —O—R¹⁰ | (b-1), |
| —S—R¹⁰ | (b-2), |
| —NR¹¹R¹² | (b-3), |

wherein
R¹⁰ is hydrogen, C₁₋₆alkyl, C₁₋₆alkylcarbonyl, aryl, arylC₁₋₆alkyl, C₁₋₆alkyloxycarbonylC₁₋₆alkyl, or a radical of formula -Alk-OR¹³ or -Alk-NR¹⁴R¹⁵;
R¹¹ is hydrogen, C₁₋₆alkyl, aryl or arylC₁₋₆alkyl;
R¹² is hydrogen, C₁₋₆alkyl, aryl, hydroxy, amino, C₁₋₆alkyloxy, C₁₋₆alkylcarbonylC₁₋₆alkyl, arylC₁₋₆alkyl, C₁₋₆alkylcarbonylamino, mono- or di(C₁₋₆alkyl)amino, C₁₋₆alkylcarbonyl, aminocarbonyl, arylcarbonyl, haloC₁₋₆alkylcarbonyl, arylC₁₋₆alkylcarbonyl, C₁₋₆alkyloxycarbonyl, C₁₋₆alkyloxyC₁₋₆alkylcarbonyl, mono- or di(C₁₋₆alkyl)aminocarbonyl wherein the alkyl moiety may optionally be substituted by one or more substituents independently selected from aryl or C₁₋₃alkyloxycarbonyl, aminocarbonylcarbonyl, mono- or di(C₁₋₆alkyl)aminoC₁₋₆alkylcarbonyl, or a radical or formula -Alk-OR¹³ or -Alk-NR¹⁴R^{15;}
wherein Alk is C₁₋₆alkanediyl;
R¹³ is hydrogen, C₁₋₆alkyl, C)₆alkylcarbonyl, hydroxyC₁₋₆alkyl, aryl or arylC₁₋₆alkyl;
R¹⁴ is hydrogen, C₁₋₆alkyl, aryl or arylC₁₋₆alkyl;
R¹⁵ is hydrogen, C₁₋₆alkyl, C₁₋₆alkylcarbonyl, aryl or arylC₁₋₆alkyl;
R⁴ is a radical of formula 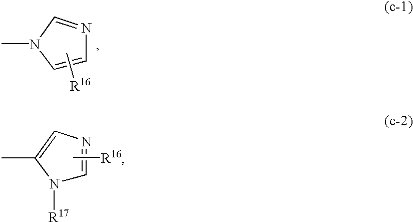
wherein
R¹⁶ is hydrogen, halo, aryl, C₁₋₆alkyl, hydroxyC₁₋₆alkyl, C₁₋₆alkyloxyC₁₋₆alkyl, C₁₋₆alkyloxy, C₁₋₆alkylthio, amino, mono- or di(C₁₋₄alkyl)amino, hydroxycarbonyl, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;
R¹⁶ may also be bound to one of the nitrogen atoms in the imidazole ring of formula (c-i) or (c-2), in which case the meaning of R¹⁶ when bound to the nitrogen is limited to hydrogen, aryl, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl or C₁₋₆alkylS(O)₂C₁₋₆alkyl;
R¹⁷ is hydrogen, C₁₋₆alkyl, C₁₋₆alkyloxyC₁₋₆alkyl, arylC₁₋₆alkyl, trifluoromethyl or di(C₁₋₄alkyl)aminosulfonyl;
R⁵ is C₁₋₆alkyl, C₁₋₆alkyloxy or halo;
aryl is phenyl, naphthalenyl or phenyl substituted with 1 or more substituents each independently selected from halo, C₁₋₆alkyl, C₁₋₆alkyloxy or trifluoromethyl

2. A combination as claimed in claim 1 wherein the farnesyl protein transferase inhibitor is a compound of formula (1) wherein X is oxygen and the dotted line represents a bond.

3. A combination as claimed in claim 1 or claim 2 wherein the farnesyl protein transferase inhibitor is a compound of formula (I) wherein R¹ is hydrogen, C₁₋₆alkyl, C₁₋₆alkyloxyC₁₋₆alkyl or mono- or di(C₁₋₆alkyl)aminoC₁₋₆alkyl and wherein R³ is hydrogen and R² is halo, C₁₋₆alkyl, C26alkenyl, C₁₋₆alkyloxy, trihalomethoxy or hydroxyC₁₋₆alkyloxy.

4. A combination as claimed in any of the preceding claims wherein the farnesyl protein transferase inhibitor is a compound of formula (I) wherein R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, C₁₋₆alkyloxyC₁₋₆alkylcarbonyl, hydroxy, or a radical of formula -Alk²-OR¹³ wherein R¹³ is hydrogen or C₁₋₆alkyl.

5. A combination as claimed in claim 1 wherein the farnesyl transferase inhibitor is selected from:
4-(3-chlorophenyl)-6-[(4-chlorophenyl)hydroxy(1-methyl-1H-imidazol-5-yl)-methyl]-1-methyl-2(1H)-quinolinone,
6-[amino(4-chlorophenyl)-1-methyl-1H-imidazol-5-ylmethyl]-4-(3-chlorophenyl)-1-methyl-2(1H)-quinolinone;
6-[(4-chlorophenyl)hydroxy(1-methyl-1H-imidazol-5-yl)methyl]-4-(3-ethoxy-phenyl)-1-methyl-2(1H)-quinolinone;
6-[(4-chlorophenyl)(1-methyl-1H-imidazol-5-yl)methyl]-4-(3-ethoxyphenyl)-1-methyl-2(1H)-quinolinone monohydrochloride.monohydrate;
6-[amino(4-chlorophenyl)(1-methyl-1H-imidazol-5-yl)methyl]-4-(3-ethoxyphenyl)-1-methyl-2(1H)-quinolinone, and
6-amino(4-chlorophenyl)(1-methyl-1H-imidazol-5-yl)methyl]-1-methyl-4-(3-propylphenyl)-2(1H)-quinolinone; a stereoisomeric form thereof or a pharmaceutically acceptable acid or base addition salts thereof.

6. A combination as claimed in claim 1 wherein the farnesyl transferase inhibitor is (+)-6-[amino(4-chlorophenyl)(1-methyl-1H-imidazol-5-yl)methyl]-4-(3-chloro-phenyl)-1-methyl-2(1H)-quinolinone; or a pharmaceutically acceptable acid addition salt thereof.

7 A combination as claimed in claim 1 wherein the farnesyl protein transferase inhibitor is a compound of formula (IX) wherein ═X¹—X²—X³ is a trivalent radical of formula (x-2), (x-3) or (x-4), >Y1-Y2 is a trivalent radical of formula (y-2), (y-3) or (y-4), r and s are 1, t is 0, R¹ is halo, preferably chloro, and most preferably 3-chloro or R¹ is C₁₋₄alkyl, preferably 3-methyl, R² is halo, preferably chloro, and most preferably 4-chloro, R³ is a radical of formula (b-1) or (b-3), R⁴ is a radical of formula (c-2), R⁶ is C₁₋₄alkyl, R⁹ is hydrogen, R¹⁰ and R¹¹ are hydrogen and R¹² is hydrogen or hydroxy.

8. A combination as claimed in claim 1 wherein the farnesyl protein transferase inhibitor is 5-(3-chlorophenyl)-α-(4-chlorophenyl)-α-(1-methyl-1H-imidazol-5-yl)tetrazolo[1,5-a]quinazoline-7-methanamine or a pharmaceutically acceptable acid addition salt thereof.

9. A combination as claimed in any of the preceding claims in which the taxane compound is paclitaxel or docetaxel.

10. A combination as claimed in any of the preceding claims in the form of a pharmaceutical composition comprising a taxane compound and a farnesyl transferase inhibitor selected from compounds of formulae (I), (II), (III), (IV), (V), (VI), (VII), (VIII) and (IX) (as defined in claim 1) together with one or more pharmaceutical carriers.

11. A combination as claimed in any of the preceding claims for use in medical therapy.

12. A combination as claimed in claim 11 for inhibiting the growth of tumor cells.

13. Use of a combination as claimed in any of claims 1 to 12 in the manufacture of a pharmaceutical composition for inhibiting the growth of tumor cells.

14. A method of inhibiting the growth of tumor cells in a human subject which comprises administering to the subject an effective amount of a combination as claimed in any of claims 1 to 12.
